(19) United States
(12) Patent Application Publication
(10) Pub. No.: US 2022/0042901 A1
(43) Pub. Date: Feb. 10, 2022

(54) Hydrogen Embrittlement Progress Evaluation Method and Hydrogen Embrittlement Progress Evaluation Device

(71) Applicant: Nippon Telegraph and Telephone Corporation, Tokyo (JP)

(72) Inventors: Takuya Kamisho, Musashino-shi, Tokyo (JP); Ryuta Ishii, Musashino-shi, Tokyo (JP); Yosuke Takeuchi, Musashino-shi, Tokyo (JP); Masayuki Tsuda, Musashino-shi, Tokyo (JP)

(21) Appl. No.: 17/275,144

(22) PCT Filed: Sep. 26, 2019
(86) PCT No.: PCT/JP2019/037857

§ 371 Date: Mar. 10, 2021

(30) Foreign Application Priority Data
Oct. 1, 2018 (JP) 2018-186637

(51) Int. Cl. G01N 17/00; G01N 33/204

(52) U.S. Cl. CPC G01N 17/006; G01N 33/204

(57) ABSTRACT

A cumulative distribution function of fracture time is calculated from large quantities of hydrogen embrittlement fracture test data measured under test conditions that assume reinforcing bars in prestressed concrete, transition probability is calculated from the cumulative distribution function of fracture time with a hydrogen embrittlement fracture being regarded as a stochastic process through which a degraded state progressively reaches a fractured state, and the time integral of the transition probability is found as the degree of progress of hydrogen embrittlement.

### TECHNICAL FIELD

The present invention relates to a hydrogen embrittlement progress evaluation method and hydrogen embrittlement progress evaluation apparatus.

### BACKGROUND ART

In recent years, there has been increasing demand for high-strength steel used for reinforcing bars in prestressed concrete as building materials. High-strength steel has high hydrogen embrittlement susceptibility, and loses ductility and gets remarkably reduced in strength if hydrogen is contained continuously in the steel. This phenomenon is called hydrogen embrittlement (see Non-Patent Literature 1). In relation to hydrogen embrittlement of steel, hydrogen embrittlement resistance is evaluated as performance of the steel. For example, an evaluation method is widely used that measures fracture time of steel by applying constant tensile stress (constant-load test) to the steel while electrochemically charging hydrogen into the steel (see Non-Patent Literature 2).

### CITATION LIST

### Non-Patent Literature

Non-Patent Literature 1: Shiraga, et al., “Hydrogen Embrittlement of Steel” Zairyo-to-Kankyo, Japan Society of Corrosion Engineering, 2011, Vol. 60, No. 5, pp. 236-420
Non-Patent Literature 2: “Method for Testing Hydrogen Embrittlement of PC Steel in a 20% Ammonium Thiocyanate Solution,” JSCE S 1201, Japan Society of Corrosion Engineering, 2012
Non-Patent Literature 3: “Introduction to Reliability Engineering (Second Revised Edition),” Marusen Publishing, 1971, p. 103

### SUMMARY OF THE INVENTION

### Technical Problem

Regarding steel actually applied to facilities and the like, if the degree of progress of hydrogen embrittlement up to when the steel fractures due to hydrogen embrittlement can be known, it becomes possible to predict to what extent hydrogen embrittlement will progress during the service period of the steel and determine to renew the facilities with an appropriate timing before the steel fractures.

However, a conventional method for evaluating hydrogen embrittlement resistance using fracture time is mainly aimed at qualitative evaluation among steels and is not able to know the degree of progress of hydrogen embrittlement up to when the steel fractures.

The present invention has been made in view of the above circumstances and has an object to know the degree of progress of hydrogen embrittlement up to when steel fractures due to hydrogen embrittlement.

### Means for Solving the Problem

A hydrogen embrittlement progress evaluation method according to the present invention comprises: a first step of finding a cumulative distribution function of fracture time from hydrogen embrittlement fracture test data measured under a test condition that assumes a reinforcing bar in prestressed concrete; a second step of calculating transition probability from the cumulative distribution function of the fracture time with a hydrogen embrittlement fracture being regarded as a stochastic process through which a degraded state progressively reaches a fracture; and a third step of finding a time integral of the transition probability as a degree of progress of hydrogen embrittlement.

A hydrogen embrittlement progress evaluation apparatus according to the present invention comprises: a cumulative distribution function calculation unit configured to find a cumulative distribution function of fracture time from hydrogen embrittlement fracture test data measured under a test condition that assumes a reinforcing bar in prestressed concrete; a transition probability calculation unit configured to calculate transition probability from the cumulative distribution function of the fracture time with a hydrogen embrittlement fracture being regarded as a stochastic process through which a degraded state progressively reaches a fracture; and a hydrogen embrittlement progress calculation unit configured to find a time integral of the transition probability as a degree of progress of hydrogen embrittlement.

### Effect of the Invention

The present invention makes it possible to know the degree of progress of hydrogen embrittlement up to when steel fractures due to hydrogen embrittlement.

### BRIEF DESCRIPTION OF DRAWINGS

FIG. 1 is a functional block diagram showing a configuration of a hydrogen embrittlement progress evaluation apparatus according to the present embodiment.

FIG. 2 is a diagram showing an example of hydrogen embrittlement fracture time data.

FIG. 3 is a diagram showing plots of hydrogen embrittlement fracture time data shown in FIG. 2 on Weibull probability paper.

FIG. 4 is a graph showing time variation in transition probability.

FIG. 5 is a graph showing time variation in the degree of progress of hydrogen embrittlement.

FIG. 6 is a graph showing time variation in transition probability found by a method different from the graph of FIG. 4.

FIG. 7 is a graph showing time variation in the degree of progress of hydrogen embrittlement calculated using the transition probability found by the different method.

FIG. 8 is a graph showing the degree of progress of hydrogen embrittlement found by combining the degrees of progress of hydrogen embrittlement found under different environmental conditions.

FIG. 9 is a graph showing transition probability found from the degree of progress of hydrogen embrittlement shown in FIG. 8.

FIG. 10 is a graph showing cumulative fracture probability predicted using the transition probability of FIG. 9 and results of an experiment actually conducted under same conditions.

FIG. 11 a flowchart showing a process flow of the hydrogen embrittlement progress evaluation apparatus according to the present embodiment.

FIG. 12 a diagram showing an exemplary hardware configuration of the hydrogen embrittlement progress evaluation apparatus.

### DESCRIPTION OF EMBODIMENTS

An embodiment of the present invention will be described below with reference to the drawings.

(Configuration of Hydrogen Embrittlement Progress Evaluation Apparatus)

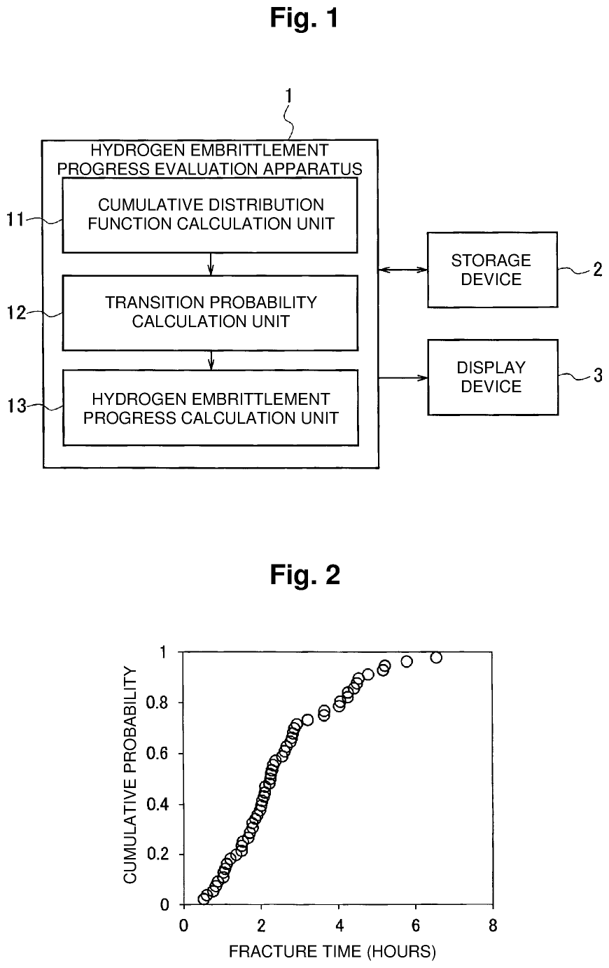

FIG. 1 is a functional block diagram showing a configuration of a hydrogen embrittlement progress evaluation apparatus 1 according to the present embodiment. The hydrogen embrittlement progress evaluation apparatus 1 shown in FIG. 1 includes a cumulative distribution function calculation unit 11, a transition probability calculation unit 12, and a hydrogen embrittlement progress calculation unit 13. The components of the hydrogen embrittlement progress evaluation apparatus 1 may be implemented by a computer equipped with an arithmetic processor and a storage device such that processes of the components will be performed by a program. The program is stored in the storage device of the hydrogen embrittlement progress evaluation apparatus and can be recorded on a recording medium such as a magnetic disk, optical disc, or semiconductor memory or provided via a network.

The hydrogen embrittlement progress evaluation apparatus 1 is connected with a storage device 2 and display device 3. Alternatively, the hydrogen embrittlement progress evaluation apparatus 1 may include the storage device 2 and display device 3.

The cumulative distribution function calculation unit 11 calculates a cumulative distribution function of fracture time from large quantities of hydrogen embrittlement fracture test data measured under test conditions that assume reinforcing bars in prestressed concrete. The hydrogen embrittlement fracture test data is stored in the storage device 2. The cumulative distribution function calculation unit 11 calculates the cumulative distribution function of fracture time by reading the hydrogen embrittlement fracture test data out of the storage device 2.

Hydrogen embrittlement fracture time data such as shown in FIG. 2 is used. The hydrogen embrittlement fracture time data shown in FIG. 2 was resulted from a large number of fracture time values acquired by repeating a test that involved measuring fracture time by applying constant tensile stress to high-strength steel while charging hydrogen into the steel. In FIG. 2, the values of fracture time are plotted in ascending order using an average ranking method. Test conditions were as follows. Smooth round bars 7 mm in diameter and 50 cm in length were used as test pieces. Tensile stress (0.9σb) was 0.9 times the tensile strength. The hydrogen charging method used was a cathodic charging method that involved immersing steel in an electrolyte aqueous solution and applying a negative potential. The electrolyte aqueous solution used was 1 mol/L of sodium bicarbonate aqueous solution plus 0.13 mol/L of ammonium thiocyanate aqueous solution. The applied potential used was −1 V vs. SSE. The number of tests was N=55. In these test conditions, reinforcing bars in prestressed concrete are assumed, and the hydrogen content in the steel was increased greatly compared to the actual environment to accelerate hydrogen embrittlement.

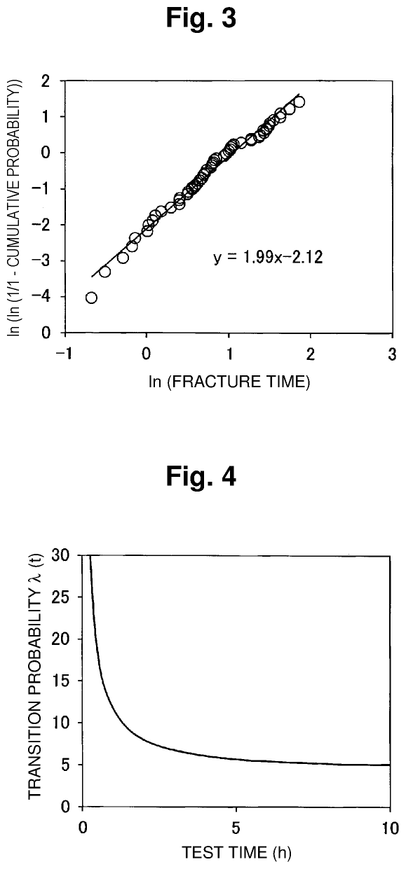

To find the cumulative distribution function of fracture time from the hydrogen embrittlement fracture time data, a typical method can be used, and no particular method is specified. The fracture time due to hydrogen embrittlement is considered to follow a Weibull distribution, and thus a cumulative distribution function F(t) is estimated using, for example, typical Weibull probability paper. When the cumulative distribution function F(t) was calculated from the hydrogen embrittlement fracture time data of FIG. 2 using Weibull probability paper as shown in FIG. 3, Formula (1) shown below was obtained.

Formula 1

F(t)=1−exp(−0.12t^{1.99}) (1)

Using the cumulative distribution function calculated by the cumulative distribution function calculation unit 11, the transition probability calculation unit 12 calculates the transition probability with a hydrogen embrittlement fracture being regarded as a stochastic process through which a degraded state progressively reaches a fractured state.

Based on the following two suppositions, the inventors found that is was possible to calculate the transition probability of a degraded state caused by hydrogen embrittlement and estimate the degree of progress of hydrogen embrittlement from the transition probability.

Supposition 1: with hydrogen embrittlement, degradation accumulates over time and a fracture occurs when the degradation exceeds a predetermined value.

Supposition 2: a change from a degraded state to another degraded state occurs probabilistically according to a transition probability λ(t).

The transition probability is the probability that a degraded state transitions once per unit time and represents the average value of degradation increase rates. That is, it is considered that a value H(t) of the time integral of the transition probability (degradation increase rate) represents the degree of progress of hydrogen embrittlement.

The idea that degradation progresses probabilistically, leading to a fracture is also applicable to fatigue failure of metallic materials. However, in the case of fatigue failure, because the transition probability λ is a constant value not subject to time variation, the idea is not directly applicable to phenomena, such as hydrogen embrittlement, whose transition probability (degradation increase rate) is considered to be subject to time variation. On the other hand, in the field of reliability engineering, in a system called a duplex system in which any devices that fail are replaced one by one by backup devices, it is known that a relationship between a failure rate λ(t) of one device and a cumulative distribution function F(t) that represents a failure rate of the entire system is known to be given by Formula (2) shown below when the number of backup devices is n (see Non-Patent Literature 3).

$$\begin{matrix}Formula 2 & \\ F ( t ) = 1 - {e}^{- H ( t )} {\sum }_{i = 0}^{n - 1} \frac{{H ( t )}^{i}}{i !} ( H ( t ) = {\int }_{0}^{t} λ ( t ) d t ) & ( 2 )\end{matrix}$$

The transition of a degraded state caused by hydrogen embrittlement is considered to be based on the same mechanism as the mechanism of transitioning to a backup device when a device fails in a duplex system. That is, the use of Formulae (1) and (2) makes it possible to find the transition probability λ(t) of hydrogen embrittlement. Also, when the cumulative distribution function F(t) follows a Weibull distribution, Formula (2) cannot be solved analytically, but can be approximated accurately if the transition probability λ(t) is given by Formula (3) shown below.

Formula 3

λ(t)=At^{-B}+C (3)

According to the above method, assuming that a fracture occurs when a degraded state transitions 100 times (n=100), if Formula (3) is substituted into Formula (2) and values of A, B, and C in Formula (3) are determined so as to fit the cumulative distribution function F(t) of Formula (1), the transition probability λ(t) is calculated as shown by Formula (4) below. The time variation graph in transition probability given by Formula (4) is shown in FIG. 4.

Formula 4

λ(t)=7.4t^{-0.91}+0.4 (4)

Note that although the value of n at which a fracture occurs is set arbitrarily, the curve of the transition probability λ(t) is almost similar even if the value of n is changed as long as n is sufficiently large, and thus for the purpose of knowing the degree of progress of hydrogen embrittlement up to when a fracture occurs, n may be any value, provided that n is sufficiently large.

The hydrogen embrittlement progress calculation unit 13 finds the time integral of the transition probability as the degree of progress of hydrogen embrittlement. No particular method is specified, and a typical method can be used. The hydrogen embrittlement progress calculation unit 13 displays the degree of progress of hydrogen embrittlement thus found on the display device 3.

The degree of progress of hydrogen embrittlement H(t) is calculated from Formula (4) as shown by Formula (5) below.

$$\begin{matrix}Formula 5 & \\ H ( t ) = {\int }_{0}^{t} λ ( t ) dt = 82.2 {t}^{0.09} + 4.0 t & ( 5 )\end{matrix}$$

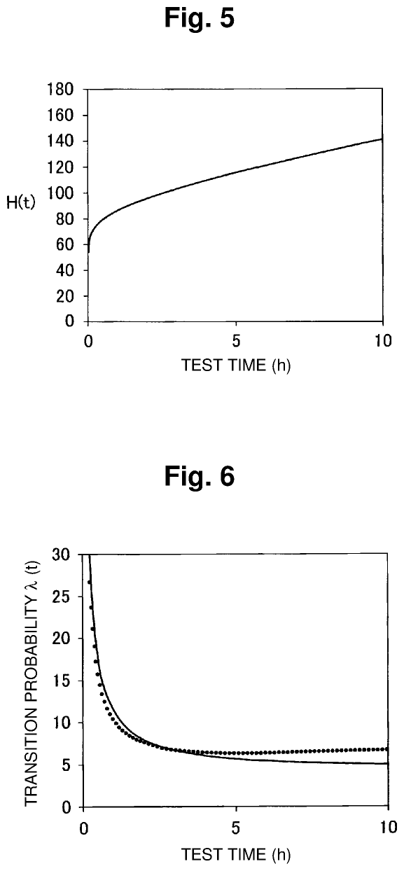

FIG. 5 shows time variation in the degree of progress of hydrogen embrittlement H(t) calculated by the hydrogen embrittlement progress evaluation apparatus 1 according to the present embodiment.

Since it is assumed that a fracture occurs when a degraded state transitions 100 times (H(t)=100), t (t=2.6 hours) at which H(t)=100 corresponds to an average fracture time. Also, when read from FIG. 5, time t at which the degree of progress of hydrogen embrittlement H(t) reaches 90% a fracture level (H(t)=90) is t=1.4 hours.

The hydrogen embrittlement progress evaluation apparatus 1 may include a renewal time estimation unit configured to associate the hydrogen embrittlement fracture test data used to calculate the degree of progress of hydrogen embrittlement with life data of steel in actual facilities and estimate a renewal time of the actual facilities from the found degree of progress of hydrogen embrittlement. For example, if average life of the steel used to acquire the hydrogen embrittlement fracture time data used this time is 20 years in an actual environment, t=2.6 hours, which satisfies H(t)=100, corresponds to 20 years in an actual environment. To renew the actual facilities when the degree of progress of hydrogen embrittlement is 90% the fracture level, the actual facilities can be renewed in the actual environment after about 11 years that correspond to t=1.4 hours at which H(t)=90.

(Another Method for Finding Transition Probability)

The transition probability λ(t) can be found more accurately using the following method. When the cumulative distribution function F(t) of fracture time in Formula (1) is substituted into Formula (2) and the formula is rearranged with respect to t, Formula (6) shown below is obtained.

$$\begin{matrix}Formula 6 & \\ t = {η ( H ( t ) + ln {\sum }_{i = 0}^{n - 1} \frac{{H ( t )}^{i}}{i !} )}^{\frac{1}{m}} & ( 6 )\end{matrix}$$

By substituting values 1 to 100 in sequence into the degree of progress of hydrogen embrittlement H(t) in Formula (6) and finding the value of t corresponding to the value of H(t), variation of H(t) with time is found. The transition probability λ(t) can be approximated by the slope of H(t). Using Formula (7) shown below, the slope of H(t) is found as λ(t) by the calculus of finite differences.

$$\begin{matrix}Formula 7 & \\ λ ( {t}_{1} ) = \frac{H ( {t}_{2} ) - H ( {t}_{1} )}{{t}_{2} - {t}_{1}} & ( 7 )\end{matrix}$$

where t1 and t2 are arbitrary times, and t1<t2. This method calculates λ(t) directly without using an approximation formula, and thus can find λ(t) more accurately.

Values of λ(t) are found by the method for finding the slope of H(t) and the method that uses the approximation formula of (4), and compared between the two methods as shown in FIG. 6, where results found by the method for finding the slope of H(t) are indicated by plots and results found by the method that uses the approximation formula of (4) are indicated by a solid line. It can be seen from FIG. 6 that the method for finding the slope of H(t) is more sensitive to accuracy differences than is the method that uses the approximation formula of (4), especially in the latter half of the testing.

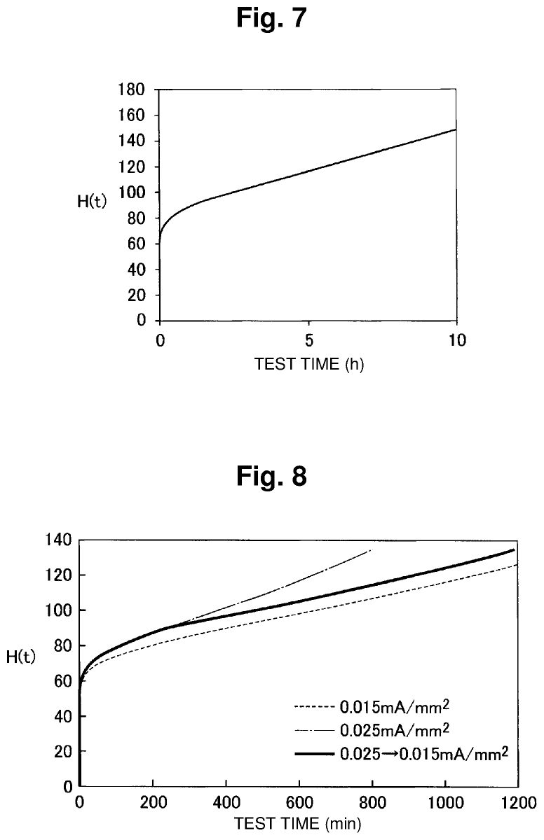

FIG. 7 shows the time variation in the degree of progress of hydrogen embrittlement H(t) calculated using λ(t) found by the method for finding the slope of H(t) on the hydrogen embrittlement progress evaluation apparatus 1. It can be seen from FIG. 7 that the average fracture time t at which H(t)=100 is 2.5 hours. Also, the time t at which H(t)=90 is 1.2 hours.

(When Environmental Conditions Change)

By combining degrees of progress of hydrogen embrittlement found under different environmental conditions the present embodiment can forecast the degree of progress of hydrogen embrittlement when environmental conditions change, such as when hydrogen generation conditions resulting from corrosion reactions or progress rates of hydrogen embrittlement vary with the seasons.

First, the degrees of progress of hydrogen embrittlement H(t) are found under different environmental conditions using the methods described so far. In FIG. 8, the alternate long and short dash line indicates the degree of progress of hydrogen embrittlement that occurs when hydrogen is charged electrochemically at 0.025 mA/mm², simulating a condition in which hydrogen embrittlement progresses quickly. The broken line indicates the degree of progress of hydrogen embrittlement that occurs when hydrogen is charged electrochemically at 0.015 mA/mm², simulating a condition in which hydrogen embrittlement progresses slowly.

H(t) exhibits behavior such as indicated by a solid line in FIG. 8 under environmental conditions in which hydrogen is charged at 0.025 mA/mm² at the start of testing and charged at 0.015 mA/mm² after a lapse of 240 minutes from the start of testing. In the example of FIG. 8, H(t) indicated by the solid line changes similarly to the degree of progress of hydrogen embrittlement at 0.025 mA/mm² until 240 minutes elapse, and changes similarly to the degree of progress of hydrogen embrittlement at 0.015 mA/mm² after a lapse of 240 minutes. When the hydrogen embrittlement progress calculation unit 13 combines the degrees of progress of hydrogen embrittlement found under different environmental conditions, it becomes possible to find the degree of progress of hydrogen embrittlement under changing environmental conditions.

Whether the degree of progress of hydrogen embrittlement is predicted correctly under changing environmental conditions can be checked by a stochastic simulation (Monte Carlo simulation). Probability P that a degraded state transitions to a next state while Δt elapses from the time t is expressed by P=λ(t)Δt using λ(t). For example, cumulative fracture probability is found by setting Δt such that P=0.01 and repeating a simulation from the start of testing to a fracture under conditions that a degraded state transitions to a next degraded state with P=0.01 while Δt elapses from the time t and that a fracture occurs when the degraded state reaches 100. When the cumulative fracture probability found by the simulation is compared with results of an experiment actually conducted under the same conditions, if the results of the simulation and experiment match each other, it can be said that the prediction about the degree of progress of hydrogen embrittlement is correct.

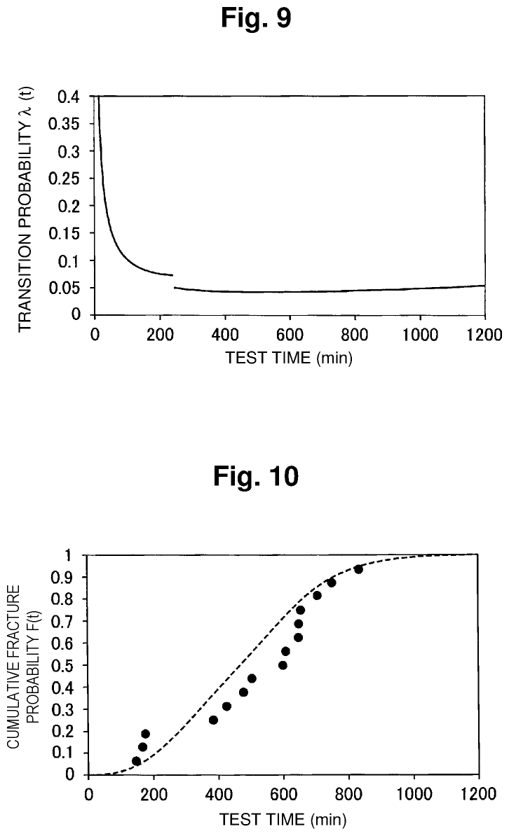

The transition probability λ(t) found from the slope of H(t) in FIG. 8 is shown in FIG. 9. The cumulative fracture probability found by repeating the simulation 1000 times using λ(t) of FIG. 9 and results of an experiment actually conducted under the same conditions are shown in FIG. 10. In FIG. 10, the simulation results are indicated by a dotted line and experimental results are indicated by plots. The cumulative fracture probability found experimentally and the cumulative fracture probability found by the simulation using λ(t) generally match each other, indicating that the experimental results can be predicted by the simulation. Thus, it can be said that the degree of progress of hydrogen embrittlement under changing environmental conditions can be predicted correctly.

In this way, if the degrees of progress of hydrogen embrittlement are grasped in a few cases of unchanging environmental conditions in advance, the degree of progress of hydrogen embrittlement under changing environmental conditions can be predicted. Because in an actual environment, environmental conditions change in many cases, this method allows the degree of progress of hydrogen embrittlement in an actual environment to be predicted using simple experiments.

(Operation of Hydrogen Embrittlement Progress Evaluation Apparatus)

Next, a process flow of the hydrogen embrittlement progress evaluation apparatus 1 according to the present embodiment will be described.

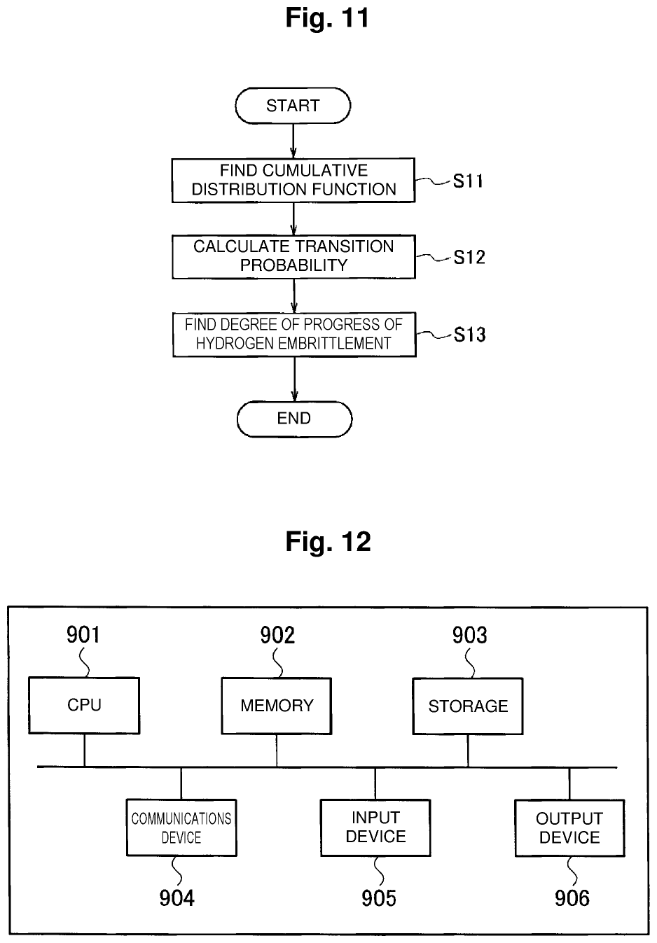

FIG. 11 a flowchart showing a process flow of the hydrogen embrittlement progress evaluation apparatus 1 according to the present embodiment. It is assumed that hydrogen embrittlement fracture test data measured under test conditions that assume reinforcing bars in prestressed concrete has already been acquired and stored in the storage device 2.

The cumulative distribution function calculation unit 11 finds a cumulative distribution function of fracture time from the hydrogen embrittlement test data (step S11).

With a hydrogen embrittlement fracture being regarded as a stochastic process through which a degraded state progressively reaches a fracture, the transition probability calculation unit 12 calculates transition probability from the cumulative distribution function of fracture time (step S12).

The hydrogen embrittlement progress calculation unit 13 finds the time integral of the transition probability as the degree of progress of hydrogen embrittlement (step S13).

If provided on the hydrogen embrittlement progress evaluation apparatus 1, the renewal time estimation unit estimates a renewal time of actual facilities from the degree of progress of hydrogen embrittlement based on life data of reinforcing bars in the actual facilities, the reinforcing bars having been used for measurement of hydrogen embrittlement fracture test data.

As has been described above, the present embodiment calculates a cumulative distribution function of fracture time from large quantities of hydrogen embrittlement fracture test data measured under test conditions that assume reinforcing bars in prestressed concrete, calculates transition probability from the cumulative distribution function of fracture time with a hydrogen embrittlement fracture being regarded as a stochastic process through which a degraded state progressively reaches a fractured state, finds the time integral of the transition probability as the degree of progress of hydrogen embrittlement, and thereby makes it possible to know the degree of progress of hydrogen embrittlement up to when steel fractures due to hydrogen embrittlement. Consequently, if the degree of progress of hydrogen embrittlement of the steel is evaluated in advance, it becomes possible to predict to what extent hydrogen embrittlement will progress during the service period of the steel and thereby provide criterion for determining the renewal time of facilities that use the steel.

For example, a general-purpose computer system such as illustrated in FIG. 12 can be used for the hydrogen embrittlement progress evaluation apparatus 1 described above, where the computer system includes a central processing unit (CPU) 901, a memory 902, a storage 903, a communications device 904, an input device 905, and an output device 906. On the computer system, as the CPU 901 executes a predetermined program loaded into the memory 902, the hydrogen embrittlement progress evaluation apparatus 1 is implemented.

### REFERENCE SIGNS LIST

1 Hydrogen embrittlement progress evaluation apparatus
11 Cumulative distribution function calculation unit
12 Transition probability calculation unit
13 Hydrogen embrittlement progress calculation unit
2 Storage device
3 Display device

1. A hydrogen embrittlement progress evaluation method comprising:
a first step of finding a cumulative distribution function of fracture time from hydrogen embrittlement fracture test data measured under a test condition that assumes a reinforcing bar in prestressed concrete;
a second step of calculating transition probability from the cumulative distribution function of the fracture time with a hydrogen embrittlement fracture being regarded as a stochastic process through which a degraded state progressively reaches a fracture; and
a third step of finding a time integral of the transition probability as a degree of progress of hydrogen embrittlement.

2. The hydrogen embrittlement progress evaluation method according to claim 1, wherein the second step finds the transition probability by approximating the transition probability with λ(t)=At^{-B}+C.

3. The hydrogen embrittlement progress evaluation method according to claim 1, wherein the second step finds a time value for each degree of progress of hydrogen embrittlement and uses a rate of change in the degree of progress of hydrogen embrittlement as the transition probability.

4. The hydrogen embrittlement progress evaluation method according to claim 1, wherein the third step further finds a degree of progress of hydrogen embrittlement under changing environmental conditions by combining degrees of progress of hydrogen embrittlement found under different environmental conditions.

5. The hydrogen embrittlement progress evaluation method according to claim 1, comprising a fourth step of estimating a renewal time of an actual facility from the degree of progress of hydrogen embrittlement based on life data of a reinforcing bar in the actual facility, the reinforcing bar being used for measurement of hydrogen embrittlement fracture test data.

6. A hydrogen embrittlement progress evaluation apparatus comprising:
a cumulative distribution function calculation unit configured to find a cumulative distribution function of fracture time from hydrogen embrittlement fracture test data measured under a test condition that assumes a reinforcing bar in prestressed concrete;
a transition probability calculation unit configured to calculate transition probability from the cumulative distribution function of the fracture time with a hydrogen embrittlement fracture being regarded as a stochastic process through which a degraded state progressively reaches a fracture; and
a hydrogen embrittlement progress calculation unit configured to find a time integral of the transition probability as a degree of progress of hydrogen embrittlement.

7. The hydrogen embrittlement progress evaluation apparatus according to claim 6, wherein the transition probability calculation unit finds the transition probability by approximating the transition probability with λ(t)=At^{-B}+C.

8. The hydrogen embrittlement progress evaluation apparatus according to claim 6, wherein the transition probability calculation unit finds a time value for each degrees of progress of hydrogen embrittlement and uses a rate of change in the degree of progress of hydrogen embrittlement as the transition probability.

9. The hydrogen embrittlement progress evaluation apparatus according to claim 6, wherein the hydrogen embrittlement progress calculation unit further finds a degree of progress of hydrogen embrittlement under changing environmental conditions by combining degrees of progress of hydrogen embrittlement found under different environmental conditions.

10. The hydrogen embrittlement progress evaluation apparatus according to claim 6, comprising a renewal time estimation unit configured to estimate a renewal time of an actual facility from the degree of progress of hydrogen embrittlement based on life data of a reinforcing bar in the actual facility, the reinforcing bar being used for measurement of hydrogen embrittlement fracture test data.
